(19) United States
(12) Patent Application Publication
(10) Pub. No.: US 2023/0287041 A1
(43) Pub. Date: Sep. 14, 2023

(54) IMPROVEMENTS TO WASH SOLUTIONS FOR ANION EXCHANGE CHROMATOGRAPHY IN A METHOD OF PURIFICATION OF RECOMBINANTLY-PRODUCED RSV PROTEINS

(71) Applicant: Pfizer Inc., New York, NY (US)

(72) Inventors: Jill Ann PADDOCK, Manchester, NH (US); Alexandra PETTAWAY, Somerville, MA (US); Jeffrey Richard SALM, Wakefield, MA (US)

(73) Assignee: Pfizer Inc., New York, NY (US)

(21) Appl. No.: 18/006,800

(22) PCT Filed: Jul. 22, 2021
(86) PCT No.: PCT/IB2021/056629

§ 371 Date: Jan. 25, 2023

Related U.S. Application Data
(60) Provisional application No. 63/056,949, filed on Jul. 27, 2020.

(51) Int. Cl. C07K 1/18; C07K 14/005

(52) U.S. Cl. CPC C07K 1/18; C07K 14/005

(57) ABSTRACT

The invention relates to a purification method of an RSV protein, wherein a load solution comprising the RSV protein is contacted with an anion exchange chromatography medium, whereby the RSV protein binds to the anion exchange chromatography medium, the anion exchange chromatography medium is washed with at least one wash solution and the RSV protein is eluted from the anion exchange chromatography medium.

REFERENCE TO SEQUENCE LISTING

This application is being filed electronically via EFS-Web and includes an electronically submitted sequence listing in .txt format. The .txt file contains a sequence listing entitled “PC072530A_SeqListing_ST25.txt” created on Jul. 1, 2021 and having a size of 1.24 MB. The sequence listing contained in this .txt file is part of the specification and is herein incorporated by reference in its entirety.

### TECHNICAL FIELD

The present invention relates to processes of manufacturing respiratory syncytial virus (RSV) vaccines. More specifically, the invention relates to methods of purification of recombinantly-produced RSV proteins including an anion exchange chromatography step.

### BACKGROUND OF THE INVENTION

Recombinant proteins such as those used for therapeutic or prophylactic purposes, are produced in genetically engineered host cells, harvested from bioreactors and then purified under controlled multi-step processes designed to confer a high degree of purity to the final product.

One of the main challenges is the reduction of impurities, in particular residual host cell proteins (HCPs), that are proteins expressed by the host cells used for the production of the therapeutic protein.

While, from a regulatory standpoint, there may not be any defined acceptable level of HCP for all the biopharmaceutical products, it is required, on a case-by-case basis, to minimize the level of HCP in order to minimize any associated safety risk and negative effect on efficacy.

One of the conventional steps involved in the purification methods for proteins for therapeutic or prophylactic use consists of an anion exchange chromatography step, wherein a load solution comprising the target protein is applied to an anion exchange chromatography medium, e.g. in the form of a resin arranged in a chromatography column.

Such an anion exchange chromatography column may be operated in a bind-and-elute mode, wherein
a load solution obtained from a harvested cell culture fluid and comprising the RSV protein is contacted with an anion exchange chromatography medium, whereby the RSV protein binds to the anion exchange chromatography medium;
the anion exchange chromatography medium is washed with at least a wash solution for the removal of impurities; and
the RSV protein is eluted from the anion exchange chromatography medium.

It is commonly expected that the protein of interest would lose binding capacity as the operating pH for the anion exchanger decreases. Wash solutions having a pH of about 7.0 or more are therefore typically used in such methods to maintain product binding. Lower pH conditions are typically not pursued for an anion exchange step because commonly admitted principles suggest that proteins will have less negative surface charges as the pH decreases, thus affecting the capacity of the protein to bind to the medium.

### SUMMARY OF THE INVENTION

The inventors have found however that proteins with sialyation in glycan profile have extra negative charges, which enable the protein to remain bound to an anion exchange medium in lower pH conditions. Sialic acid content, as part of glycan modifications to the protein, has been found to increase the total amount of surface charges and maintain protein binding at lower pH ranges.

The inventors have also found that the pH condition was a significant factor of HCP reduction and that using wash solutions at lower pH conditions showed effective removal of host cell proteins.

According to a first aspect of the present invention, the anion exchange chromatography medium is washed with at least one lower pH wash solution at a pH between 3.0 and 6.5, whereby the removal of host cell proteins is enhanced.

According to preferred embodiments of the invention:
the pH of the load solution is between 7.0 and 8.5, preferably of about 7.5;
the pH of said lower pH wash solution is between 4.0 and 6.0, preferably between 4.5 and 5.5, preferably of about 5.0;
said lower pH wash solution comprises acetate;
the concentration of acetate in said lower pH wash solution is between 56 and 84 mM, preferably between 63 and 77 mM, preferably of about 70 mM;
prior to eluting the RSV protein, the anion exchange chromatography medium is further washed with at least a first higher pH wash solution at a pH between 7.0 and 8.0, preferably of about 7.5;
said first higher pH wash solution comprises Tris at a concentration between 18 and 22 mM, preferably of about 20 mM;
said first higher pH wash solution comprises NaCl at a concentration between 45 and 55 mM, preferably of about 50 mM;
the wash step using said first higher pH wash solution is performed prior to the wash step using said lower pH wash solution;
prior to the elution of the RSV protein, the anion exchange chromatography medium is further washed with at least a second higher pH wash solution at a pH between 7.0 and 8.0, preferably of about 7.5;
said second higher pH wash solution comprises Tris at a concentration between 45 and 55 mM, preferably of about 50 mM;
said second higher pH wash solution comprises NaCl at a concentration between 18 and 22 mM, preferably of about 20 mM;
the wash step using said second higher pH wash solution is performed after the wash step using said lower pH wash solution. Such further wash step enables product elution at a constant pH;
the RSV protein is eluted with an elution solution having a pH between 7.0 and 8.0, preferably of about 7.5;
said elution solution comprises NaCl at a concentration between 146 and 180 mM, preferably of about 163 mM;
said elution solution comprises Tris at a concentration between 18 and 22 mM, preferably of about 20 mM;
the load challenge is comprised between 7.5 and 15.0 mg per ml of the anion exchange chromatography medium;
the method further comprises a cHA chromatography step;
the method further comprises a HIC chromatography step;
said anion exchange chromatography, cHA chromatography and HIC chromatography steps are performed sequentially in this order;
the RSV protein is a protein from RSV subgroup A or RSV subgroup B;
the RSV protein is an RSV F protein;
the RSV F protein is in a prefusion conformation;
the RSV F protein is a mutant of a wild-type F protein for any RSV subgroup that contains one or more introduced mutations;
RSV F mutant is stabilized in prefusion conformation;
the RSV F mutant specifically binds to antibody D25 or AM-14;
the RSV protein is formulated for use as an injectable pharmaceutical product.

In a further aspect of the invention, it is provided a pharmaceutical product including an RSV protein purified by a method according to the first aspect of the invention.

In an embodiment, the recombinantly-produced RSV protein purified according to the method of the invention is an RSV F protein.

In an embodiment, the recombinantly-produced RSV protein purified according to the method of the invention is an RSV F protein from RSV subgroup A.

In an embodiment, the recombinantly-produced RSV protein purified according to the method of the invention is an RSV F protein from RSV subgroup B.

In an embodiment, the recombinantly-produced RSV protein purified according to the method of the invention is an RSV F protein in prefusion conformation.

In an embodiment, the recombinantly-produced RSV protein purified according to the method of the invention is an RSV F mutant protein.

In an embodiment, the recombinantly-produced RSV protein purified according to the method of the invention is an RSV F mutant protein stabilized in prefusion conformation.

In an embodiment, the recombinantly-produced RSV protein purified according to the method of the invention is an RSV F mutant protein in trimeric form.

In a preferred embodiment, the recombinantly-produced RSV protein purified according to the method of the invention is an RSV F mutant protein in trimeric form and stabilized in prefusion conformation.

In a most preferred embodiment, the recombinantly-produced RSV protein purified according to the method of the invention is an RSV F mutant protein in trimeric form comprising a trimerization domain linked to the C-terminus of F1 polypeptide of said F mutant protein and stabilized in prefusion conformation.

In a particular embodiment, said trimerization domain is a T4 fibritin foldon domain.

In a particular embodiment, said T4 fibritin foldon domain has the amino acid sequence

(SEQ ID NO: 40)
GYIPEAPRDGQAYVRKDGEWVLLSTFL.

In a preferred embodiment, the recombinantly-produced RSV protein purified according to the method of the invention is an RSV F mutant which specifically binds to antibody D25 and/or AM-14. Preferably the recombinantly-produced RSV protein purified according to the method of the invention is an RSV F mutant which specifically binds to antibody D25 and AM-14.

The amino acid sequence of a large number of native RSV F proteins from different RSV subtypes, as well as nucleic acid sequences encoding such proteins, is known in the art. For example, the sequence of several subtype A, B, and bovine RSV F0 precursor proteins are set forth in WO 2017/109629, SEQ ID NOs: 1, 2, 4, 6 and 81-270, which are set forth in the Sequence Listing submitted herewith. Any reference to SEQ ID NOs in the specification is to those in WO 2017/109629, which are included in the Sequence Listing contained in the .txt file submitted as part of this specification and which Sequence Listing is herein incorporated by reference in its entirety.

The native RSV F protein exhibits remarkable sequence conservation across RSV subtypes. For example, RSV subtypes A and B share 90% sequence identity, and RSV subtypes A and B each share 81% sequence identify with bovine RSV F protein, across the F0 precursor molecule. Within RSV subtypes the F0 sequence identity is even greater; for example, within each of RSV A, B, and bovine subtypes, the RSV F0 precursor protein has about 98% sequence identity. Nearly all identified RSV F0 precursor sequences consist of 574 amino acids in length, with minor differences in length typically due to the length of the C-terminal cytoplasmic tail. Sequence identity across various native RSV F proteins is known in the art (see, for example, WO 2014/160463). To further illustrate the level of the sequence conservation of F proteins, non-consensus amino acid residues among F0 precursor polypeptide sequences from representative RSV A strains and RSV B strains are provided in Tables 17 and 18 of WO 2014/160463, respectively (where non-consensus amino acids were identified following alignment of selected F protein sequences from RSV A strains with ClustalX (v. 2)).

In some specific embodiments, the recombinantly-produced RSV protein purified according to the method of the invention is an RSV F mutant comprising a pair of cystine mutations, termed “engineered disulfide bond mutation” in WO 2017/109629, wherein the mutant comprises the same introduced mutations that are in any of the exemplary mutants provided in Tables 1 and 4-6 of WO 2017/109629. The exemplary RSV F mutants provided in Tables 1 and 4-6 of WO 2017/109629 are based on the same native F0 sequence of RSV A2 strain with three naturally occurring substitutions at positions 102, 379, and 447 (SEQ ID NO:3). The same introduced mutations in each of the mutants can be made to a native F0 polypeptide sequence of any other RSV subtype or strain to arrive at different RSV F mutants, such as a native F0 polypeptide sequence set forth in any of the SEQ ID NOs: 1, 2, 4, 6, and 81-270. RSV F mutants that are based on a native F0 polypeptide sequence of any other RSV subtype or strain and comprise any of the engineered disulfide mutations are also within the scope of the invention. In some particular embodiments, the recombinantly-produced RSV protein purified according to the method of the invention is an RSV F protein mutant comprising at least one engineered disulfide mutation selected from the group consisting of: 55C and 188C; 155C and 290C; 103C and 148C; and 142C and 371C, such as S55C and L188C, S155C and S290C, T103C and I148C, or L142C and N371C.

In other embodiments, the recombinantly-produced RSV protein purified according to the method of the invention is an RSV F mutant that comprise one or more cavity filling mutations. The term “cavity filling mutation” refers to the substitution of an amino acid residue in the wild-type RSV F protein by an amino acid that is expected to fill an internal cavity of the mature RSV F protein. In one application, such cavity-filling mutations contribute to stabilizing the pre-fusion conformation of a RSV F protein mutant. The cavities in the pre-fusion conformation of the RSV F protein can be identified by methods known in the art, such as by visual inspection of a crystal structure of RSV F in a pre-fusion conformation, or by using computational protein design software (such as BioLuminate™ [BioLuminate, Schrodinger LLC, New York, 2015], Discovery Studio™ [Discovery Studio Modeling Environment, Accelrys, San Diego, 2015], MOE™ [Molecular Operating Environment, Chemical Computing Group Inc., Montreal, 2015], and Rosetta™ [Rosetta, University of Washington, Seattle,) 2015]). The amino acids to be replaced for cavity-filling mutations typically include small aliphatic (e.g. Gly, Ala, and Val) or small polar amino acids (e.g. Ser and Thr). They may also include amino acids that are buried in the pre-fusion conformation but exposed to solvent in the post-conformation. The replacement amino acids can be large aliphatic amino acids (Ile, Leu and Met) or large aromatic amino acids (His, Phe, Tyr and Trp). For example, in several embodiments, the RSV F protein mutant includes a T54H mutation.

In some specific embodiments, the recombinantly-produced RSV protein purified according to the method of the invention is an RSV F protein mutant comprising one or more cavity filling mutations selected from the group consisting of:
1) substitution of S at positions 55, 62, 155, 190, or 290 with I, Y, L, H, or M;
2) substitution of T at position 54, 58, 189, 219, or 397 with I, Y, L, H, or M;
3) substitution of G at position 151 with A or H;
4) substitution of A at position 147 or 298 with I, L, H, or M;
5) substitution of V at position 164, 187, 192, 207, 220, 296, 300, or 495 with I, Y, H; and
6) substitution of R at position 106 with W.

In some specific embodiments, the recombinantly-produced RSV protein purified according to the method of the invention is an RSV F mutant comprising one or more cavity filling mutations, wherein the mutant comprises the cavity filling mutations in any of the mutants provided in Tables 2, 4, and 6 of WO 2017/109629. RSV F mutants provided in those Tables 2, 4, and 6 are based on the same native F0 sequence of RSV A2 strain with three naturally occurring substitutions at positions 102, 379, and 447 (SEQ ID NO:3). The same introduced mutations in each of the mutants can be made to a native F0 polypeptide sequence of any other RSV subtype or strain to arrive at different RSV F mutants, such as a native F0 polypeptide sequence set forth in any of the SEQ ID NOs:1, 2, 4, 6, and 81-270. The RSV F mutants that are based on a native F0 polypeptide sequence of any other RSV subtype or strain and comprise any of the one or more cavity filling mutations are also within the scope of the invention. In some particular embodiments, the recombinantly-produced RSV protein purified according to the method of the invention is an RSV F protein mutant comprising at least one cavity filling mutation selected from the group consisting of: T54H, S190I, and V296I.

In still other embodiments, the recombinantly-produced RSV protein purified according to the method of the invention is an RSV F protein mutant including one or more electrostatic mutations. The term “electrostatic mutation” refers to an amino acid mutation introduced to a wild-type RSV F protein that decreases ionic repulsion or increase ionic attraction between residues in a protein that are proximate to each other in the folded structure. As hydrogen bonding is a special case of ionic attraction, electrostatic mutations may increase hydrogen bonding between such proximate residues. In one example, an electrostatic mutation may be introduced to improve trimer stability. In some embodiments, an electrostatic mutation is introduced to decrease repulsive ionic interactions or increase attractive ionic interactions (potentially including hydrogen bonds) between residues that are in close proximity in the RSV F glycoprotein in its pre-fusion conformation but not in its post-fusion conformation. For example, in the pre-fusion conformation, the acidic side chain of Asp486 from one protomer of the RSV F glycoprotein trimer is located at the trimer interface and structurally sandwiched between two other acidic side chains of Glu487 and Asp489 from another protomer. On the other hand, in the post-fusion conformation, the acidic side chain of Asp486 is located on the trimer surface and exposed to solvent. In several embodiments, the RSV F protein mutant includes an electrostatic D486S substitution that reduces repulsive ionic interactions or increases attractive ionic interactions with acidic residues of Glu487 and Asp489 from another protomer of RSV F trimer. Therefore, in an embodiment, the recombinantly-produced RSV protein purified according to the method of the invention comprises an electrostatic D486S substitution. Typically, introduction of an electrostatic mutation will increase the melting temperature (Tm) of the pre-fusion conformation or pre-fusion trimer conformation of the RSV F protein.

Unfavorable electrostatic interactions in a pre-fusion or pre-fusion trimer conformation can be identified by method known in the art, such as by visual inspection of a crystal structure of RSV F in a pre-fusion or pre-fusion trimer conformation, or by using computational protein design software (such as BioLuminate™ [BioLuminate, Schrodinger LLC, New York, 2015], Discovery Studio™ [Discovery Studio Modeling Environment, Accelrys, San Diego, 2015], MOE™ [Molecular Operating Environment, Chemical Computing Group Inc., Montreal, 2015.], and Rosetta™ [Rosetta, University of Washington, Seattle, 2015.]).

In some specific embodiments, the recombinantly-produced RSV protein purified according to the method of the invention is an RSV F protein mutant comprising at least one electrostatic mutation selected from the group consisting of:
1) substitution of E at position 82, 92, or 487 by D, F, Q, T, S, L, or H;
2) substitution of K at position 315, 394, or 399 by F, M, R, S, L, I, Q, or T;
3) substitution of D at position 392, 486, or 489 by H, S, N, T, or P; and
4) substitution of R at position 106 or 339 by F, Q, N, or W.

In some specific embodiments, the recombinantly-produced RSV protein purified according to the method of the invention is an RSV F mutant comprising one or more electrostatic mutations, wherein the mutant comprises the electrostatic mutations in any of the mutants provided in Tables 3, 5, and 6 of WO 2017/109629. RSV F mutants provided in those Tables 3, 5, and 6 are based on the same native F0 sequence of RSV A2 strain with three naturally occurring substitutions at positions 102, 379, and 447 (SEQ ID NO:3). The same introduced mutations in each of the mutants can be made to a native F0 polypeptide sequence of any other RSV subtype or strain to arrive at different RSV F mutants, such as a native F0 polypeptide sequence set forth in any of the SEQ ID NOs:1, 2, 4, 6, and 81-270. RSV F mutants that are based on a native F0 polypeptide sequence of any other RSV subtype or strain and comprise any of the one or more electrostatic mutations are also within the scope of the invention. In some particular embodiments, the recombinantly-produced RSV protein purified according to the method of the invention is an RSV F protein mutant comprising mutation D486S. B-2 (d) Combination of Engineered Disulfide Bond Mutations, Cavity Filling Mutations, and Electrostatic Mutations.

In another aspect, the recombinantly-produced RSV protein purified according to the method of the invention is an RSV F protein mutant comprising a combination of two or more different types of mutations selected from engineered disulfide bond mutations, cavity filling mutations, and electrostatic mutations, each as described herein above. In some embodiments, the mutants comprise at least one engineered disulfide bond mutation and at least one cavity filling mutation. In some specific embodiments, the RSV F mutants include a combination of mutations as noted in Table 4 of WO 2017/109629.

In some further embodiments, the recombinantly-produced RSV protein purified according to the method of the invention is an RSV F protein mutant comprising at least one engineered disulfide mutation and at least one electrostatic mutation. In some specific embodiments, the RSV F mutants include a combination of mutations as noted in Table 5 of WO 2017/109629.

In still other embodiments, the recombinantly-produced RSV protein purified according to the method of the invention is an RSV F protein mutant comprising at least one engineered disulfide mutation, at least one cavity filling mutation, and at least one electrostatic mutation. In some specific embodiments, the RSV F mutants include a combination of mutations as provided in Table 6 of WO 2017/109629.

In some particular embodiments, the recombinantly-produced RSV protein purified according to the method of the invention is an RSV F mutant that comprises a combination of mutations selected from the group consisting of:
(1) combination of T103C, I148C, S190I, and D486S;
(2) combination of T54H S55C L188C D486S;
(3) combination of T54H, T103C, I148C, S190I, V296I, and D486S;
(4) combination of T54H, S55C, L142C, L188C, V296I, and N371C;
(5) combination of S55C, L188C, and D486S;
(6) combination of T54H, S55C, L188C, and S190I;
(7) combination of S55C, L188C, S190I, and D486S;
(8) combination of T54H, S55C, L188C, S190I, and D486S;
(9) combination of S155C, S190I, S290C, and D486S;
(10) combination of T54H, S55C, L142C, L188C, V296I, N371C, D486S, E487Q, and D489S; and
(11) combination of T54H, S155C, S190I, S290C, and V296I.

In some specific embodiments, the RSV F mutant comprises a combination of introduced mutations, wherein the mutant comprises a combination of mutations in any of the mutants provided in Tables 4, 5, and 6 of WO 2017/109629. RSV F mutants provided in those Tables 4, 5, and 6 are based on the same native F0 sequence of RSV A2 strain with three naturally occurring substitutions at positions 102, 379, and 447 (SEQ ID NO:3). The same introduced mutations in each of the mutants can be made to a native F0 polypeptide sequence of any other RSV subtype or strain to arrive at different RSV F mutants, such as a native F0 polypeptide sequence set forth in any of the SEQ ID NOs:1, 2, 4, 6, and 81-270. RSV F mutants that are based on a native F0 polypeptide sequence of any other RSV subtype or strain and comprise any of the combination of mutations are also within the scope of the invention.

In some other particular embodiments, the recombinantly-produced RSV protein purified according to the method of the invention is an RSV F mutant comprising a cysteine (C) at position 103 (103C) and at position 148 (148C), an isoleucine (I) at position 190 (190I), and a serine (S) at position 486 (486S), and wherein the mutant comprises a F1 polypeptide and a F2 polypeptide selected from the group consisting of:
(1) a F2 polypeptide comprising the amino acid sequence of SEQ ID NO:41 and a F1 polypeptide comprising the amino acid sequence of SEQ ID NO:42;
(2) a F2 polypeptide comprising an amino acid sequence that is at least 97%, 98% or 99% identical to the amino acid sequence of SEQ ID NO:41 and a F1 polypeptide comprising an amino acid sequence that is at least 97%, 98%, or 99% identical to the amino acid sequence of SEQ ID NO:42;
(3) a F2 polypeptide comprising the amino acid sequence of SEQ ID NO: 43 and a F1 polypeptide comprising the amino acid sequence of SEQ ID NO:44;
(4) a F2 polypeptide comprising an amino acid sequence that is at least 97%, 98% or 99% identical to the amino acid sequence of SEQ ID NO:43 and a F1 polypeptide comprising an amino acid sequence that is at least 97%, 98%, or 99% identical to the amino acid sequence of SEQ ID NO:44;
(5) a F2 polypeptide comprising the amino acid sequence of SEQ ID NO: 45 and a F1 polypeptide comprising the amino acid sequence of SEQ ID NO:46;
(6) a F2 polypeptide comprising an amino acid sequence that is at least 97%, 98% or 99% identical to the amino acid sequence of SEQ ID NO:45 and a F1 polypeptide comprising an amino acid sequence that is at least 97%, 98%, or 99% identical to the amino acid sequence of SEQ ID NO:46;
(7) a F2 polypeptide comprising the amino acid sequence of SEQ ID NO: 47 and a F1 polypeptide comprising the amino acid sequence of SEQ ID NO:48;
(8) a F2 polypeptide comprising an amino acid sequence that is at least 97%, 98% or 99% identical to the amino acid sequence of SEQ ID NO:47 and a F1 polypeptide comprising an amino acid sequence that is at least 97%, 98%, or 99% identical to the amino acid sequence of SEQ ID NO:48;
(9) a F2 polypeptide comprising the amino acid sequence of SEQ ID NO: 49 and a F1 polypeptide comprising the amino acid sequence of SEQ ID NO:50;
(10) a F2 polypeptide comprising an amino acid sequence that is at least 97%, 98% or 99% identical to the amino acid sequence of SEQ ID NO:49 and a F1 polypeptide comprising an amino acid sequence that is at least 97%, 98%, or 99% identical to the amino acid sequence of SEQ ID NO:50.
(11) a F2 polypeptide comprising the amino acid sequence of SEQ ID NO:279 and a F1 polypeptide comprising the amino acid sequence of SEQ ID NO:280;
(12) a F2 polypeptide comprising an amino acid sequence that is at least 97%, 98% or 99% identical to the amino acid sequence of SEQ ID NO:279 and a F1 polypeptide comprising an amino acid sequence that is at least 97%, 98%, or 99% identical to the amino acid sequence of SEQ ID NO:280;
(13) a F2 polypeptide comprising the amino acid sequence of SEQ ID NO:281 and a F1 polypeptide comprising the amino acid sequence of SEQ ID NO:282;
(14) a F2 polypeptide comprising an amino acid sequence that is at least 97%, 98% or 99% identical to the amino acid sequence of SEQ ID NO:281 and a F1 polypeptide comprising an amino acid sequence that is at least 97%, 98%, or 99% identical to the amino acid sequence of SEQ ID NO:282;
(15) a F2 polypeptide comprising the amino acid sequence of SEQ ID NO:283 and a F1 polypeptide comprising the amino acid sequence of SEQ ID NO:284;
(16) a F2 polypeptide comprising an amino acid sequence that is at least 97%, 98% or 99% identical to the amino acid sequence of SEQ ID NO:283 and a F1 polypeptide comprising an amino acid sequence that is at least 97%, 98%, or 99% identical to the amino acid sequence of SEQ ID NO:284;
(17) a F2 polypeptide comprising the amino acid sequence of SEQ ID NO:285 and a F1 polypeptide comprising the amino acid sequence of SEQ ID NO:286;
(18) a F2 polypeptide comprising an amino acid sequence that is at least 97%, 98% or 99% identical to the amino acid sequence of SEQ ID NO:285 and a F1 polypeptide comprising an amino acid sequence that is at least 97%, 98%, or 99% identical to the amino acid sequence of SEQ ID NO:286;
(19) a F2 polypeptide comprising the amino acid sequence of SEQ ID NO:287 and a F1 polypeptide comprising the amino acid sequence of SEQ ID NO:288;
(20) a F2 polypeptide comprising an amino acid sequence that is at least 97%, 98% or 99% identical to the amino acid sequence of SEQ ID NO:287 and a F1 polypeptide comprising an amino acid sequence that is at least 97%, 98%, or 99% identical to the amino acid sequence of SEQ ID NO:288;
(21) a F2 polypeptide comprising the amino acid sequence of SEQ ID NO:289 and a F1 polypeptide comprising the amino acid sequence of SEQ ID NO:290; and
(22) a F2 polypeptide comprising an amino acid sequence that is at least 97%, 98% or 99% identical to the amino acid sequence of SEQ ID NO:289 and a F1 polypeptide comprising an amino acid sequence that is at least 97%, 98%, or 99% identical to the amino acid sequence of SEQ ID NO:290.

In some specific embodiments, a trimerization domain is linked to the C-terminus of F1 polypeptide of the F mutant protein. In a particular embodiment, the trimerization domain is a T4 fibritin foldon domain, such as the amino acid sequence

(SEQ ID NO: 40)
GYIPEAPRDGQAYVRKDGEWVLLSTFL.

### DETAILED DESCRIPTION OF THE INVENTION

### Definitions

The following definitions will be used in the present description and claims:
the term “harvested cell culture fluid” (or “harvested CCF”) refers to a solution containing at least one target substance which is sought to be purified from other substances also present. The harvested CCFs are often complex mixtures containing many biological molecules (such as proteins, antibodies, hormones, and viruses), small molecules (such as salts, sugars, lipids, etc.) and even particulate matter. While a typical harvested CCF of biological origin may be an aqueous solution or suspension, it may also contain organic solvents used in earlier separation steps such as solvent precipitations, extractions, and the like. Examples of harvested CCFs that may contain valuable biological substances amenable to the purification by various embodiments of the present invention include, but are not limited to, a culture supernatant from a bioreactor, a homogenized cell suspension, plasma, plasma fractions, and milk;
the term “load” refers to any material containing the target substance, either derived from the cell culture (the harvested CCF) or from a chromatography step (thus partially purified), and loaded onto a chromatography medium;
the term “load challenge” refers to the total mass of substance loaded onto the chromatography medium in the load cycle of a chromatography step, measured in units of mass of substance per unit volume of medium;
the term “impurities” refers to materials in the harvested CCF that are different from the protein of interest (or target protein) and are desirably excluded from the final therapeutic protein formulation. Typical impurities include nucleic acids, proteins (including HCPs and low molecular weight species, peptides, endotoxins, viruses and small molecules;
the term “drug substance” refers to the therapeutic protein as an active pharmaceutical ingredient as obtainable by the processes of the present invention;
the term “drug product” refers to a finished dosage form that contains the therapeutic protein in association with excipients;
the term “excipients” means the constituents of the final therapeutic protein formulation, which are not the therapeutic protein. The excipients typically include protein stabilizers, surfactants, amino-acids e.g. contributing to protein stabilization, etc. . . ;
unless stated otherwise, the term “about” associated with a numeral value means within a range of ±5% of said value.

By “reduction of HCPs” or “enhancing the removal of HCPs”, it is meant that the concentration of HCP species present compared to the therapeutic protein is reduced in the eluted pool. The general reduction in HCPs can be measured by methods known in the art, such as HCP ELISA (usually used as the primary tool) and LC-MS/MS.

### EXAMPLE

The invention will now be further illustrated by the following Example, corresponding to a purification process applied to a recombinantly-produced RSV protein and evaluated with various AEX chromatography wash strategies. The Example is provided for illustrative purpose only and should not be construed as limiting the scope of the invention.

In the illustrative Example, the RSV protein is either an RSV A protein or an RSV B protein.

In this Example, the RSV protein, present in a load solution collected from a bioreactor, is purified by a multi-step purification process that sequentially includes
an initial centrifugation and depth filtration step;
a first ultrafiltration/diafiltration step;
an anion exchange (AEX) chromatography step, that is run in a chromatography column comprising the following medium: Fractogel™ EMD TMAE HiCap (M) available from Millipore Sigma. Alternative media may be used, such as a Q Sepharose™, Capto™ Q or Capto Q ImpRes™ resin, available from GE Healthcare, a TOYOPEARL GigaCap™ Q-650M resin from Tosoh Bioscience, or a or Eshmuno™ Q resin from Millipore Sigma. The resin is initially equilibrated with an equilibration buffer. The column is then loaded such that the target protein binds to the resin. The resin is subsequently washed with one or more wash solution(s) and an elution buffer is then applied, whereby the target protein is eluted from the resin in an elution pool. The primary objective of the AEX chromatography step is the separation of the RSV proteins from process-derived impurities;
a carbonate-containing hydroxyapatite (cHA) chromatography step, such as CHT™ Ceramic Hydroxyapatite Type I 40 μm, available from Bio-Rad;
a hydrophobic interaction chromatography (HIC) step, that is run in a chromatography column comprising a medium such as Butyl Sepharose 4 Fast Flow™ resin, available from GE Healthcare;
a virus filtration step using a ViresolvePro™ filter available from Millipore Sigma. Alternatively, a Planova™ filter, available from Asahi Kasei, may be used for this virus filtration step;
a second ultrafiltration/diafiltration step; and
a final formulation and filtration step.

Anion Exchange Chromatography—Wash Buffer Evaluation

The load solution including the target protein (RSV A or RSV B) and having a pH of 7.5±0.2 is loaded into the Fractogel™ EMD TMAE HiCap (M) column. The column is equilibrated with the following equilibration buffer: 20 mM Tris, 50 mM NaCl pH 7.5. The experiment is run with various load challenge conditions and the column is washed with various wash solutions, as reflected in Table 1 below, before elution of the protein with a 20 mM Tris, 163 mM NaCl pH 7.5 elution buffer.

The protein of interest is in a trimeric form (“Trimer” in Table 1). The “Control pH 7.5 Wash” referred to in Table 1 is a Tris, NaCl wash solution at pH 7.5.

TABLE 1

| | Resin Challenge | Trimer | Product | Pool HCP | |
|---|---|---|---|---|---|
| Run | (mg/mL) | Purity (%) | Losses (%) | (mg/L) | HCP LRV |
| Common Load Source | | 36 | | 589 | |
| Control pH 7.5 Wash | 15 | 72 | 0 | 244 | 0.5 |
| 70 mM Acetate pH 4.8 | 8 | 89 | 1 | 97 | 1.2 |
| 70 mM Acetate pH 4.8 | 18 | 86 | 16 | 190 | 0.8 |

It can be observed from this experiment that lower pH wash conditions, as compared to more conventional wash solutions at pH of 7.5, can enhance HCP reduction (by ˜1 log) with minimal product losses. Such lower pH wash conditions also increase trimer purity (86 and 89% vs. 72%).

The experiment suggests that, in the lower pH wash conditions (pH 4.8 in this Example), the product loss during the low pH wash increases as the load challenge (“Resin Challenge” in Table 1) increases.

Further experiments conducted on acetate wash solutions at various pH, acetate concentrations and load challenges suggest that: lower wash pH correlates with better HCP removal. Lower mass challenge correlates with better HCP removal and better yields. Higher acetate concentration correlates with lower yields.

The results indicate that, among the tested factors, the pH condition is the most significant factor of HCP reduction and that the buffer strength is the most significant factor for product recovery.

Beyond acetate which was used in the above described experiments, alternative anions solution may be used for this method: citrate, phosphate, sulfate, chloride.

Further experiments have been conducted to evaluate the impact of various buffer species with different anion strengths and characterize optimal conditions for HCP removal.

Table 2 below shows the wash conditions (salts, concentration, pH) which were evaluated.

TABLE 2

| | | | | | | | | | Sodium | | Sodium | |
|---|---|---|---|---|---|---|---|---|---|---|---|---|
| Salt | Sodium Citrate | | | | Sodium Acetate | | | | Phosphate | | Sulfate | |
| Concentration | 20 | 20 | 20 | 20 | 20 | 20 | 20 | 20 | 20 | 20 | 20 | 20 |
| (mM) | 30 | 30 | 30 | 30 | 30 | 30 | 30 | 30 | 30 | 30 | 30 | 30 |
| | 50 | 50 | 50 | 50 | 50 | 50 | 50 | 50 | 50 | 50 | 50 | 50 |
| | 70 | 70 | 70 | 70 | 70 | 70 | 70 | 70 | 70 | 70 | 70 | 70 |
| | 90 | 90 | 90 | 90 | 90 | 90 | 90 | 90 | 90 | 90 | 90 | 90 |
| | 110 | 110 | 110 | 110 | 110 | 110 | 110 | 110 | 110 | 110 | 110 | 110 |
| | 130 | 130 | 130 | 130 | 130 | 130 | 130 | 130 | 130 | 130 | 130 | 130 |
| | 150 | 150 | 150 | 150 | 150 | 150 | 150 | 150 | 150 | 150 | 150 | 150 |
| pH | 3.5 | 4 | 4.5 | 5 | 3.5 | 4 | 4.5 | 5 | 3.5 | 5 | 3.5 | 5 |

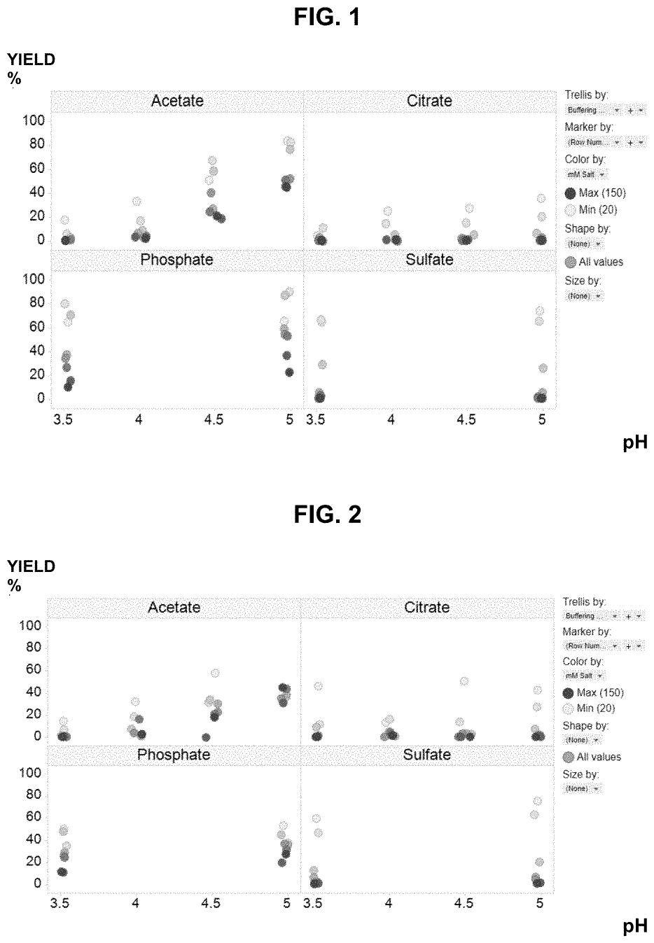

FIG. 1 and FIG. 2 plot the yield values (percentage of product recovery) obtained for each wash solution, against the pH value, respectively for an RSV A and an RSV B load solution.

It will be observed on FIG. 1, for RSV A, that
(i) an increased buffer strength leads to lower yields due to product losses during low pH wash;
(ii) acetate and citrate show correlation of yield with wash pH;
(iii) phosphate and sulfate show higher yields at pH 3.5 and are less sensitive to pH.

Data presented on FIG. 2, for RSV B, suggest that
(i) a similar trend as for RSV A is observed with acetate i.e. lower pH and increased strength leading to lower yields;
(ii) yield is not as sensitive to high buffer strength as for RSV A;
(iii) lower recoveries are observed for RSVB as compared to RSV A.

In a further experiment, the performance of multiple wash conditions (buffer type, concentration, pH) for both RSV A and RSV B was evaluated in terms of HCP reduction and yield, and compared to the preferred wash solution: 70 mM Acetate, pH 5.0.

The data generated have been collated in Table 3 below.

TABLE 3

| | | | | | | |
|---|---|---|---|---|---|---|
| | | | | | Current process | |
| | | | | | condition in | Expected |
| | | | Yields | HPC removal | HTS screen: | based on |
| | Concentration | pH | observed in | logs of | 70 Mm Acetate | historical |
| Buffer Type | Range (mM) | Range | screen (%) | Removal (LRV) | pH 5 | process data |

RSVA [PF-06934186]

| | | | | | | |
|---|---|---|---|---|---|---|
| Acetate | 20-110 | 5 | 51-83 | 0.7-0.8 | Yield: 51% | Yield: 70% |
| | 20-50 | 4.5 | 51-59 | 0.8 | HCP | HCP Removal: |
| Phosphate | 20-50 | 3.5 | 65-80 | 0.6-0.7 | Removal: ~0.8LRVs | 0.9-1.1 LRVs |
| | 20-110 | 5 | 53-90 | 0.6-0.9 | | |
| Sulfate | 20-30 | 3.5-5 | 65-74 | 0.5-0.6 | | |

RSVB [PF-06937100]

| | | | | | | |
|---|---|---|---|---|---|---|
| Citrate | 20 | 4.5-5 | 42-50 | 0.8-1.0 | Yield: 38% | Yield: 65% |
| Acetate | 20 | 4.5 | 58 | 0.8 | HCP | Removal: |
| | 110-150 | 5 | 44-45 | 0.8-0.9 | Removal: ~0.8LRVs | 0.9-1.4 LRVs |
| Phosphate | 20 | 3.5-5 | 50-53 | 0.6 | | |
| | 50 | 3.5-5 | 45-48 | 0.7 | | |
| Sulfate | 20-30 | 3.5-5 | 47-75 | 0.5 | | |

In Table 3, the data obtained for the preferred wash solution (70 mM Acetate, pH 5.0) with a high throughput screening (HTS) method—as shown in the penultimate column—have been normalized based on historical data and show:
for RSV A: a yield of 70% and a log reduction value (LRV) of HCP between 0.9 and 1.1; and
for RSV B: a yield of 65% and an LRV between 0.9 and 1.4.

The conducted wash screens suggest that increased buffer strengths result in yield losses during wash and decreased wash pH result in better HCP removal. RSV A and RSV B showed similar trends with yield and HCP, with RSV B showing lower yields. Different buffers showed a range of effectiveness between HCP removal and yield, in particular phosphate and sulfate which are robust options as alternatives to acetate based on normalized data in Table 3.

Finally, with a load solution having a pH between 7.0 and 8.5, and more specifically of about 7.5, and a load challenge comprising between 7.5 and 15.0 mg per ml of the anion exchange chromatography medium, the preferred low pH wash conditions for the AEX chromatography column applicable to both RSV A and RSV B is: 70 mM Acetate and pH 5.0.

Based on the aforementioned experiments, an acceptable range of pH for the low pH wash solution may be between 3.0 and 6.5, more preferably between 4.0 and 6.0, and most preferably between 4.5 and 5.5.

With these operating conditions, phosphate and sulfate are robust options as alternatives to acetate.

In the actual method, prior to loading the load solution including the target protein (RSV A or RSV B) into the AEX column, the column is equilibrated with an equilibration solution: 20 mM Tris, 50 mM NaCl pH 7.5.

After loading, the column is successively washed with three wash solutions, the second one being the lower pH wash solution, the first and third ones being the higher pH wash solutions:
Wash #1: 20 mM Tris, 50 mM NaCl, pH 7.5;
Wash #2: 70 mM Acetate, pH 5.0;
Wash #3: 50 mM Tris, 20 mM NaCl, pH 7.5.

The aforementioned pH values and compositions for the wash solutions are those preferred, however acceptable performances in terms of HCP reduction and yield may also be obtained under the following conditions:
the first higher pH wash solution (Wash #1) may have a pH between 7.0 and 8.0. Tris concentration may be between 18 and 22 mM and NaCl concentration may be between 45 and 55 mM;
the concentration of acetate in the lower pH wash solution (Wash #2) may be between 56 and 84 mM, more preferably between 63 and 77 mM. The acceptable ranges of pH, as discussed above, are 3.0-6.5, preferably 4.0-6.0, and more preferably 4.5-5.5;
the second higher pH wash solution (Wash #3) may have a pH between 7.0 and 8.0. Tris concentration may be between 45 and 55 mM and NaCl concentration may be between 18 and 22 mM.

After the washing step performed by washing the column successively with the three wash solutions, the RSV protein is eluted with an elution solution. The elution solution comprises NaCl at a concentration between 146 and 180 mM, preferably of about 163 mM, and Tris at a concentration between 18 and 22 mM, preferably of about 20 mM. The pH of the elution solution is between 7.0 and 8.0, and is preferably of about 7.5.

The subsequent chromatography steps of the Example are preferably operated in the following conditions.

cHA Chromatography

Prior to loading the product into the cHA chromatography column, the column is equilibrated with a first equilibration buffer 0.5 M sodium phosphate, pH 7.2 and then with a second equilibration buffer 20 mM Tris, 100 mM NaCl, 13 mM sodium phosphate, pH 7.0.

The product pool collected from the AEX chromatography column and adjusted with phosphate addition, after filtration, is loaded into the cHA chromatography column. The pH of the load is set at a value of 7.1±0.3 and the load challenge is comprised between 8.0 and 12.0 mg per ml of medium.

The column is washed with a wash solution comprising: 20 mM Tris, 100 mM NaCl, 13 mM sodium phosphate, pH 7.0.

The column is operated in a flow-through mode, meaning that, as the load fluid is loaded into the column, the target protein flows through the column while the impurities bind to the medium. The wash is intended to wash the unintentionally bound target proteins out of the column.

HIC

Prior to loading the product into the HIC column, the column is equilibrated with a first equilibration buffer comprising 20 mM potassium phosphate at pH 7.0, and then with a second equilibration buffer comprising 1.1 M potassium phosphate at pH 7.0.

The product pool collected from the cHA chromatography column and adjusted with potassium phosphate addition, after filtration, is loaded into the HIC column. The pH and the conductivity of the load are adjusted to respectively 7.0±0.3 and 104±10 mS/cm. The load challenge comprises between 8.0 and 12.0 mg per ml of medium.

The column is operated in a bind and elute mode, whereby the target proteins loaded into the column bind to the medium and then are eluted by applying an elution buffer. Before applying the elution buffer, the column is washed with a wash solution in order to wash out impurities bound to the medium.

The wash solution used in this HIC step is 1.1 M potassium phosphate, pH 7.0 and the elution buffer is 448 mM potassium phosphate, pH 7.0.

The above-described method is suitable for purifying recombinantly-produced RSV proteins with a sufficient degree of purity, such that said proteins may be used for the preparation of pharmaceutical products. In particular, such purified RSV proteins may be formulated, by addition of suitable excipients, for use as an injectable pharmaceutical product.

1. Method of purification of a recombinantly-produced RSV protein, comprising an anion exchange chromatography step wherein
a) a load solution obtained from a harvested cell culture fluid and comprising the RSV protein is contacted with an anion exchange chromatography medium, whereby the RSV protein binds to the anion exchange chromatography medium;
b) the anion exchange chromatography medium is washed with at least one lower pH wash solution at a pH between 3.0 and 6.5, whereby the removal of host cell proteins is enhanced; and
c) the RSV protein is eluted from the anion exchange chromatography medium.

2. Method according to claim 1, wherein the pH of the load solution is between 7.0 and 8.5.

3. Method according to claim 1 or 2, wherein the pH of said lower pH wash solution is between 4.0 and 6.0.

4. Method according to claim 1, wherein said lower pH wash solution comprises acetate.

5. Method according to claim 4, wherein the concentration of acetate in said lower pH wash solution is between 56 and 84 mM.

6. Method according to claim 1, wherein prior to eluting the RSV protein, the anion exchange chromatography medium is further washed with at least a first higher pH wash solution at a pH between 7.0 and 8.0.

7. Method according to claim 6, wherein said first higher pH wash solution comprises Tris at a concentration between 18 and 22 mM.

8. Method according to claim 6 or 7, wherein said first higher pH wash solution comprises NaCl at a concentration between 45 and 55 mM.

9. Method according to claim 6, wherein the wash step using said first higher pH wash solution is performed prior to the wash step using said lower pH wash solution.

10. Method according to claim 6, wherein prior to the elution of the RSV protein, the anion exchange chromatography medium is further washed with at least a second higher pH wash solution at a pH between 7.0 and 8.0.

11. Method according to claim 10, wherein said second higher pH wash solution comprises Tris at a concentration between 45 and 55 mM.

12. Method according to claim 10 or 11, wherein said second higher pH wash solution comprises NaCl at a concentration between 18 and 22 mM.

13. Method according to claim 10, wherein the wash step using said second higher pH wash solution is performed after the wash step using said lower pH wash solution.

14. Method according to claim 1, wherein the RSV protein is eluted with an elution solution having a pH between 7.0 and 8.0.

15. Method according to claim 14, wherein said elution solution comprises NaCl at a concentration between 146 and 180 mM.

16. Method according to claim 14, wherein said elution solution comprises Tris at a concentration between 18 and 22 mM.

17. Method according to claim 1, wherein the load challenge is between 7.5 and 15.0 mg per ml of the anion exchange chromatography medium.

18. Method according to claim 1, further comprising a cHA chromatography step.

19. Method according to claim 1, further comprising a HIC chromatography step.

20-28. (canceled)

29. Pharmaceutical product including an RSV protein purified by a method according to claim 1.

30-37. (canceled)
